(19) United States
(12) Patent Application Publication
(10) Pub. No.: US 2008/0112908 A1
(43) Pub. Date: May 15, 2008

(54) Anti-fungal nail lacquer

(76) Inventor: David B. Srulevitch, Walnut, CA (US)

Correspondence Address: Boniard I. Brown, 1710 West Cameron Ave., #200, West Covina, CA, 91790, US

(21) Appl. No.: 11/599,555

(22) Filed: Nov. 15, 2006

(51) Int. Cl. A61K 8/36

(52) U.S. Cl. 424/61

(57) ABSTRACT

An anti-fungal nail lacquer composition contains a film-forming agent, a solvent, pigments and/or dyes, a glacial acetic acid of appropriate concentration dissolved in the composition. A method of utilizing the anti-fungal lacquer comprises the application of the composition to a fingernail or toenail, and allowing it to remain in contact with the nail until the solvents evaporate and the film of glacial acetic acid remains on the nail.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND AND SUMMARY OF THE INVENTION

The present invention relates to a fingernail lacquer having an anti-fungal effective amount of glacial acetic acid. Conventionally, nail lacquer compositions contain film-forming compounds, organic solvents and/or dyes and pigments. Dyes and pigments serve to cover an unsightly yellow-brown appearance of a nail caused by fungal infections. The dyes/pigments are at least partially suspended in the nail lacquer, and typically contains a suspending agent such as bentonite.

Fingernails and toenails are susceptible to dermatophytic infections caused by the invasion of fungi into the nails. There are numerous fungi, including T. rubrum, T. mentagrophytum, and T. interdigitale, and other known fungi that causes these types of infections. Treatment of these infections may typically involve the administering of one or more types of anti-fungal agents—e.g., griseofulvin, clotrimazole, miconazole nitrate and thiabendazole, either taken orally or topically, depending upon the particular anti-fungal agent used.

These compounds, due to their insolubility and associated poor adsorption from the gastrointestinal tract, have to be administered in relatively large doses, for long periods of time, sometimes extending for more than one year, and have a series of undesirable side effects. An effective topical formulation will be advantageous over an oral formulation, because it will minimize the risk of undesirable side effects. A drawback of some topically applied formulations is that the drug may not remain on the nail and may be easily removed by rubbing or brushing against clothing, shoes, etc.

A soluble anti-fungal agent formulation in a nail lacquer will not present this problem, since the nail-lacquer acts as a barrier relative to the environment once it has dried.

Another drawback of most topically applied anti-fungal agents is lack of patient compliance. Unsightly discoloration of the nails, caused by the fungal infection, will remain until the infection is cleared up, which may require more than six months of treatment. During such period of time, the patient may easily become discouraged and discontinue treatment, thus either not curing the infection or causing a relapse. A colored anti-fungal nail lacquer has been developed which colors the nail with an aesthetically pleasing formulation which encourages rather than detracts from patient compliance.

### BRIEF DESCRIPTION OF THE SEVERAL VIEWS OF THE DRAWINGS

Not applicable.

### DETAILED DESCRIPTION OF THE INVENTION

The present invention is based upon the discovery that addition of glacial acetic acid to a nail lacquer and the application of the lacquer to toenails or fingernails, the glacial acetic acid effectively prevents and/or alleviates infections of the nails to which the anti-fungal nail lacquer is applied.

Generally, the nail lacquer according to the invention would include organic film former in solution in a solvent system of one or more biocompatible organic solvents which, upon being applied to the nails, evaporate, leaving a relatively hard water permeable film. Both the film forming compounds and the solvents systems are well known in the art.

Nail lacquer formulations also may include a plasticizer to render the film more elastic. Plasticizers are generally known in the cosmetic art and include camphor, diphenyl and dibutyl phthalate, and tricresyl and triphenyl phosphate. If desired, other ingredients may include dyes and pigments such as titanium dioxide, mica, bismuth oxychloride, Red 6 Lake, Iron Oxides, Yellow 5 Lake, etc. If insoluble dyes and pigments are utilized, a suspending agent such as bentonite should be added to the nail lacquer composition, thus to prevent settling out of such insoluble ingredients.

The quantity of glacial acetic acid in the nail lacquer compositions according to the present invention is generally between 5 weight percent and 50 weight percent, a preferred amount being between 10 weight percent and 20 weight percent with all the glacial acetic acid in solution.

One embodiment of the present invention relates to a composition which comprises at least 10 weight percent in a clear lacquer which is essentially similar in composition to that used in manicured nails. This can be topically applied to infected sites of nails and surrounding tissue, which exhibit signs of fungal infection. The invention is also directed to methods of making and utilizing the composition.

Once the glacial acetic acid, containing lacquer, is applied to the nail it rapidly hardens and forms a water-permeable film containing the glacial acetic acid after the evaporation of the solvent system. This concentrates the glacial acetic acid at or near where it is needed, and is considered to facilitate the transport of the active compound through the patient's nail. The nail lacquer composition of the invention provides means for applying a concentrated amount of an active compound, but also promotes transport of the compound through the nail. The above description relative to the mechanism of absorption is a theory based upon observed facts, and should not in any way limit the scope of the present invention.

The nail lacquer composition may be prepared by mixing a clear or colored nail lacquer with the active compound in an appropriate manner. The composition may be applied to the affected area by any suitable means, such as a brush of the type utilized for manicuring nails.

The composition according to the invention may be utilized to treat many types of dermathophytic infections. The composition may be used to treat nail infections caused by fungi (Onychomycosis). Such infections may be caused by any of numerous fungi, which are classified under the genera: trichophyton, microsporum or epidermophyton.

A preferred embodiment of the anti-fungal agent glacial acetic acid utilized to treat fungal infections in accordance with this embodiment of the invention is according to the following formula:

| Compound | Parts by Weight |
|---|---|
| Ethyl Acetate | 34.90 |
| Butyl Acetate | 27.00 |
| Nitrocellulose | 12.00 |
| Isopropyl Alcohol | 10.00 |
| Phthallic Anhydride/Trimellitic | 5.00 |
| Anhydride Glycols Copolymer | |
| Dibutyl Phthalate | 2.00 |
| Styrene/Acrylates Copolymer | 1.50 |
| Stearalkonium Hectorite | 1.20 |
| Stearalkonium Bentonite | 1.20 |
| Camphor | 1.20 |
| Bismuth Oxychloride | 2.40 |
| Red Iron Oxide | 0.50 |
| Red 6 Lake | 0.40 |
| Red 34 Lane | 0.40 |
| Benzophenone-1 | 0.10 |
| Fragrance | 0.20 |

To the above suspension base, 20 weight percent of glacial acetic acid is added, and is mixed with a variable speed laboratory stirrer until a uniform suspension is obtained.

A clear nail lacquer can also be prepared in a similar manner according to the above formulation, but lacking the dyes and pigments utilized to prepare the colored formulation.

The resulting composition is applied directly to the infected nail. After being applied, the solvents quickly evaporate because of the body temperature of the patient, and leaves as a residue a thin film on the affected area.

The composition may be applied to the affected area by any appropriate means, a small brush perhaps being the most satisfactory. The effective amount of composition applied to the infected area provides a thin layer of the residue after evaporation of the solvents. Precise amount of the composition applied is not critical, but excessive application is not beneficial. Removal of the lacquer should be done periodically by using a suitable solvent, typically every two to four days. This can be accomplished with a nail polish remover commercially available or preferably by a mixture of acetone containing 5 weight percent glacial acetic acid in solution. This maintains the appropriate levels of the drug in the tissue.

A preferred method for utilizing the composition is to apply two coats of the colored nail lacquer containing 20 weight percent of glacial acetic acid, followed by two coats of clear nail lacquer which also contains 20 weight percent of glacial acetic acid, once a day for two consecutive days. On the third day, the nails and surrounding areas are cleaned using a medicated nail polish remover containing 5 weight percent glacial acetic acid. The process of nail lacquer application is then repeated for a period of six months or until the nails are completely clear of infection. This process may require three to six months depending on the seriousness of the infection, although complete resolution may occur within four months in some patients.

While the preferred embodiments have been described, various modifications and substitutions may be made without departing from the spirit and scope of the invention. It will be understood by those skilled in the art that any commercial nail polish, colored or clear, can be used in the practice of the invention. Accordingly, it is to be understood that the present invention has been described by way of illustration and not limitation.

The inventor claims:

1. An anti-fungal nail lacquer composition comprising:
an organic film former in an organic solvent system therefor,
glacial acetic acid in appropriate concentration,
a suspending agent, and
said organic film former forming a water-permeable film containing the glacial acetic acid upon the evaporation of the organic solvent system.

2. An anti-fungal nail lacquer composition according to claim 1, wherein the weight ratio of film former to organic solvent is about 1:3 to 1:10.

3. A composition according to claim 2 wherein the amount of glacial acetic acid is between 0.5 weight percent to 10 weight percent and the combined weight of film former and organic solvent is between about 70 weight percent to 99.5 weight percent.

4. A composition according to claim 1 wherein a suspending agent is bentonite.

5. A composition according to claim 1 wherein said film former comprises nitrocellulose.

6. A method for the treating of dermatophytic infections of the nails of humans and animals, comprising the application to said nails of an anti-fungal quantity of a nail lacquer composition comprising essentially a film former in an organic solvent system, a suspending agent, glacial acetic acid, and allowing said nail lacquer composition to remain on said nails until sufficient organic solvent has evaporated to leave on said nails a water-permeable film comprising said film-former and said glacial acetic acid.

7. A method according to claim 6 wherein the ratio by weight of film-former to organic solvent in the nail lacquer composition is from 1:3 to 1:10.

8. A method according to claim 6 wherein the amount of glacial acetic acid is between 5 percent by weight to 50 percent by weight and the combined weight of the film former and organic solvent is between 70 weight percent and 99.5 weight percent in the nail lacquer composition.

9. A method according to claim 6 wherein the suspending agent is bentonite.

10. A method according to claim 6 wherein said film-former is nitrocellulose.
